(19) United States
(12) Patent Application Publication
(10) Pub. No.: US 2015/0139853 A1
(43) Pub. Date: May 21, 2015

(54) METHOD AND APPARATUS FOR TRANSFORMING A LIQUID STREAM INTO PLASMA AND ELIMINATING PATHOGENS THEREIN

(71) Applicant: Alfredo ZOLEZZI-GARRETON, Vina del Mar (CL)

(72) Inventor: Alfredo ZOLEZZI-GARRETON, Vina del Mar (CL)

(73) Assignee: AIC, LLC, Dover, DE (US)

(21) Appl. No.: 14/084,622

(22) Filed: Nov. 20, 2013

(51) Int. Cl. A61L 2/14

(52) U.S. Cl. CPC A61L 2/14
USPC 422/22; 422/186.23

(57) ABSTRACT

A liquid stream is transformed into a vapor and liquid medium, then plasma state is generated in the medium, which generates various high-energy particles causing a number of physical and/or chemical effects, then the vapor and liquid medium is condensed back into an output stream of liquid. Liquid feedstock (e.g. water) is nebulized using pressure drop and/or acoustic waves within a chamber, then an electric field exceeding the breakdown voltage threshold of the nebulized medium is applied to the medium, thus igniting plasma state. At the exit of the chamber, the nebulized medium is transformed back into liquid state. Liquid treatment with plasma state applications are thus enabled with high versatility and control in igniting and maintaining a plasma state at a cost-effective level of energy consumption.

### FIELD OF THE INVENTION

The invention relates to a method of applying plasma particles to a liquid stream. More specifically, the invention relates to a method and apparatus for generating plasma by ionizing particles of a liquid stream that has been transformed into a liquid-and-gas biphasic medium. The plasma particles, thus generated, are utilized to treat the liquid to achieve various results among which: disabling microorganisms, promoting chemical reactions, separating one or more compounds and synthesizing new compounds.

### BACKGROUND OF THE INVENTION

Plasma is a state of matter that is composed of charged particles exhibiting collective behavior. Since the particles in plasma are electrically charged, generally by being stripped of electrons, it is considered as an ionized gas. Free electrical charges (not bound to atoms or ions) cause plasma to be electrically conductive.

Plasma state can be artificially generated by large electrical discharges confined within a small space. Using plasma, a substantial amount of energy can be applied at a high level of spatial control, thus, several industrial applications that require localized application of high energy utilize plasma. Plasma discharges are used in the treatment of solids, liquids and gases. For example, in engineering and construction, plasma is widely used for welding under seawater using arc discharges in aqueous electrolytes.

The characteristic feature of arc discharge in liquid media is the formation of a plasma discharge region close to the electrode ends. In recent years, electric arc discharge in water has been used in several physico-chemical studies, and in the production of certain materials.

There are a number of examples of attempts to generate plasma within a volume of liquid. A number of patents and published patent applications describe methods and apparatuses for the initiation of plasma discharges within a contained liquid volume, where gaseous bubbles are present, and for the use of this discharge for stimulation of chemical processes such as the decomposition of compounds and cracking of materials, which may be used in detoxification.

In US patent (U.S. Pat. No. 7,067,204), Nomura et al. (2006) disclose a “Submerged plasma generator, method of generating plasma in liquid and method of decomposing toxic substance with plasma in liquid”. The apparatus includes an ultrasonic wave generator for generating bubbles in the liquid, and an electromagnetic wave generator for continuously irradiating electromagnetic waves into the liquid from within the liquid in order to generate plasma. The method of generating plasma in a liquid includes the steps of generating bubbles in the liquid by irradiating ultrasonic waves in the liquid, and generating plasma in the bubbles by continuously irradiating electromagnetic waves from within the liquid to the bubbles. This invention comprises various methods for generating the bubbles inside the liquid medium, such as a heating device, a decompression device or an ultrasonic wave generator. The gas-liquid ratio achieved by the latter described bubble generating method is small. Basically, the liquid phase prevails in the medium. Therefore, the steady burning zone of the discharge is quite small, resulting in a very small field of applications for the device.

In US patent (U.S. Pat. No. 5,270,515), Long and Raymond (1993), disclose a “Microwave plasma detoxification reactor and process for hazardous wastes”. In the latter patent, a large volume microwave plasma process for “in-situ” detoxification of dioxins, furans and other toxicants is disclosed. A helical coil and a cylinder of low loss dielectric tubing are coaxially positioned inside a microwave resonant cavity to extend from a cross-polarized fluid inlet to a cross-polarized vapor outlet. Fluid passing through the coil cylinder is directly ionized to the plasma state by microwave energy introduced into the cavity. The geometry of the coil relative to the cylinder induces a magnetic field in the plasma compressing the plasma to the center of the cylinder, thereby preventing charring of the cylinder walls. Said geometry also provides a slower fall through rate for the treatment of liquid and solid waste. The process and apparatus are particularly suitable for mobile applications, for on-site treatment of hazardous wastes. In the latter apparatus, a liquid medium is treated by the microwave irradiation for ionization. However, the latter method requires complicated equipment and high energy microwave irradiation and can be applied only for a restricted range of liquids. In the latter invention microwaves are used to ionize the fluid passing through the coil cylinder for producing plasma, which consumes large amounts of energy.

In US patent (U.S. Pat. No. 4,886,001), Chang et al. disclose a “Method and apparatus for plasma pyrolysis of liquid waste”. The method is characterized by injecting a mixture of waste and water into a plasma torch having an operation temperature over 5000° C. to form a mixture of product gases and solid particulate. The gases and particulate are separated in a cyclone separator. A second cyclone separator and a partial vacuum separate any carryover gases from the particulate. The carryover gases and the particulate are treated in a scrubber with a caustic solution and water in order to eliminate any carryover particulate from the gases, and to neutralize hydrochloric acid (HCl) present in the gases. Finally the gases are removed from the scrubber. In the latter apparatus, plasma is only used as a high temperature source, used for decomposition of substances.

In US patent (U.S. Pat. No. 5,603,895), Martens et al. describe a “Plasma water vapor sterilizer and method”. The apparatus for plasma sterilization of articles has a plasma generator, a sterilizing chamber and a source of water vapor in fluid communication with the plasma generator. The method for plasma sterilization comprises exposing an article to be sterilized to a neutral active species of plasma, generated from water vapor. The exposure of the article to the plasma is carried out at reduced pressures and a chamber temperature of less than about 82° C. for a time period sufficient to effect sterilization.

In US patent (U.S. Pat. No. 7,931,811), titled “Dielectric barrier reactor having concentrated electric field”, Ruan et al. disclose a method of treating liquids by generating an electric field across a gap between two electrodes, concentrating the electric field within the gap by dividing the gap with a dielectric separator, which comprises an electric field passageway extending through the separator from a first gap to a second gap. The liquid passes through the electric field passageway during the step of generating the electric field.

Considering the significant advantages of using plasma to produce several effects on liquids, such as inducing chemical reactions to both break down compounds and synthesize new ones, it is highly desirable to have methods and apparatuses that operate on continuous mode, with high intensity, at a cost-effective level and under operational requirements favorable for industrial and household applications.

### SUMMARY OF THE INVENTION

The invention provides a method and apparatus for transforming a liquid stream into a stable plasma discharge. The methods of generating and maintaining plasma, according to the invention, rely on vaporizing a liquid stream to obtain a gas-and-liquid biphasic medium within a reaction chamber, then delivering an electrical stimulation to initiate plasma state in the biphasic medium. The methods of the invention are quick to initiate plasma state, and are highly efficient in power consumption to initiate and maintain a plasma state.

The vaporization of the liquid may be achieved by nebulization. Droplets of the nebulized liquid range in diameter from a few nanometers to a few micrometers. The liquid-gas medium within the chamber is suitable for generating plasma by ionization (e.g., by applying an electric discharge). The ionized particles remain inside the plasma reaction chamber for a certain amount of time, and serve to maintain the plasma state.

In one or more applications of the invention, the following stage comprises reversing the liquid-gas phase back to a liquid phase. Furthermore, one or more separation stages of byproducts may be carried out simultaneously or successively to separate one or more gases, solid particles or any other byproduct that have been produced as a byproduct of the plasma state within the chamber.

The method according to implementations of the invention involves nebulizing a liquid stream, which may be achieved by transitioning the stream from a high-pressure zone (e.g., inside a pipeline) into a lower pressure zone (e.g., plasma chamber). Additionally, the transitioning may be carried out through a diaphragm, an ultrasonic transducer, an ultrasonic hydrodynamic transducer or any other means capable of nebulizing a liquid.

The methods of the invention involve ionizing the particles of the nebulized liquid stream, generating fully ionized plasma, highly ionized plasma or weakly ionized plasma. In one or more applications of the invention, ionization of the liquid-gas phase is achieved using one or more of several means, such as applying an electric field the voltage of which exceeds the breakdown voltage threshold of the nebulized liquid medium (capacitively coupled plasma), and/or applying a strong alternating magnetic field inside the nebulized liquid medium which induces electric currents (inductively coupled plasma), and/or applying microwave radiation inside the liquid-gas medium, which induces currents and electric fields in the liquid-gas medium (microwave excited plasma).

The energy consumption rate is considerably lower compared with prior art for igniting and sustaining plasma in order to apply plasma particles to a liquid. The high efficiency of the process, according to the invention, gives implementations of the invention, the flexibility, scalability and therefore modularity, all features that facilitate industrial implementation for mass-production.

It is noteworthy that prior methods that use plasma for treating liquids, generally consists of exposing the treated liquid to a source of plasma that is generated in a gaseous phase independently from the treated liquid. The method of the invention generate plasma by ionizing the particles of the treated liquid that has been transformed into a liquid-and-gas biphasic medium.

The reaction is typically carried out in a reactor that has a nozzle to atomize the liquid at the ingress, and a back pressure system at the egress. The nebulized liquid stream, in which the plasma is produced, has a dynamic cluster structure. The latter is utilized, for example, to control chemical reactions produced in the chamber by varying the pressure in the input of the nozzle and counter pressure in the outlet pipeline, which changes the regime of steady burning of the plasma and, accordingly, the direction and velocity of the chemical reactions.

An apparatus according to the invention may be utilized in a variety of systems for carrying out several applications. The disclosure describes an application for disinfecting a water supply by transforming it into plasma. The various ionic particles created in the plasma, newly synthesized molecules (e.g. Ozone), and molecules resulting from the breakdown of larger molecules may be efficient at inactivating biological agents contaminating a water stream.

In many situations of emergency, such as in the aftermath of hurricanes, monsoons, an earth quake, a flood, a terrorist attack, a war or any other affection of the kind, the water supply may become contaminated with harmful biological agents. In these cases, the invention provides a water sanitation system that can be installed on location to disinfect water from any available water source. The locations may include housing buildings, factories, hospitals or any type of building that may be, for example, a target of a terrorist attack involving hazardous biological agents. A water sanitization system embodying the invention may be placed after the water matrix and inside each building's dependencies to provide water disinfection.

A water sanitization apparatus implementing the invention is highly adaptable and versatile. For instance, a plurality of apparatuses may be combined to increase the sanitization throughput. Also, the possibility of controlling the input parameters enables a user of the apparatus to govern the generation of each of the effects over the water (i.e. UV, IR, ozone, electromagnetic fields, frequency of elastic waves), so as to optimize the disinfection process. Furthermore, the treated water may be re-circulated within the same device and/or several devices (e.g., mounted in series) in order to assure a high level of disinfection. For example, since the feedstock may contain several contaminants, each of which may require a specific treatment, a re-circulation stage may be necessary to rid the water of particular contaminant.

Moreover, because of the low energy requirement to operate an apparatus of the invention, the apparatus may be powered by a solar energy source, enabling deployment and autonomous operation in remote locations.

The invention provides numerous applications that rely on the initiation and maintenance of the plasma state as disclosed. The latter involves transforming a liquid phase into a liquid-gas phase containing droplets of liquid suspended in a gas. The particles in the liquid-gas medium are ionized, which supports the initiation and maintenance of a plasma state. This, contrary to the prior art where the plasma is generated separately from a liquid, the liquid to be treated is then exposed to the plasma discharge. Consequently, for each target effect, an implementation of the invention produces a stronger effect than any other prior art technology, at a lower energy requirement rate.

### BRIEF DESCRIPTION OF THE DRAWINGS

FIG. 1 is a flowchart diagram that represents the overall steps for transforming a liquid stream into a plasma channel and then back to liquid state, in accordance with an embodiment of the invention.

FIG. 2 is a block diagram representing the basic components of a system implementing the invention for treating a liquid stream with plasma particles, in accordance with an embodiment of the invention.

FIG. 3 shows a cut-through representation of a portion of a system for generating capacitively coupled plasma from a liquid stream in accordance with an embodiment of the invention, using internal and external electrodes.

FIG. 4A shows a cut-through representation of a portion of a system for generating capacitively coupled plasma from a liquid stream in accordance with an embodiment of the invention, using an internal inlet electrode.

FIG. 4B shows a cut-through representation of a portion of a system for generating capacitively coupled plasma from a liquid stream in accordance with an embodiment of the invention, using internal inlet and outlet electrodes.

FIG. 5 shows a cut-through representation of a portion of a system for generating capacitively coupled plasma from a liquid stream in accordance with an embodiment of the invention, using an external electrode.

FIG. 6 is a flowchart diagram representing steps of disinfecting water in accordance with an embodiment of the invention.

FIG. 7 is a block diagram representing components of a system embodying the invention to provide water disinfection.

### DETAILED DESCRIPTION OF THE INVENTION

The invention provides a method and apparatuses for transforming a liquid of an input stream into a liquid-gas stream, initiating a plasma state into the liquid-gas stream, then reversing the liquid-gas stream back into an output liquid stream. Embodiments of the invention achieve the latter by efficiently producing nebulized liquid media, which is conducive to initiating and sustaining plasma state. The invention provides means for controlling the size of the droplets, as well as the intensity of the plasma, its localization and numerous other parameters that allow one with ordinary skills in the art to apply the invention to a variety of applications for treating a liquid with plasma particles.

In the following description, numerous specific details are set forth to provide a more thorough description of the invention. It will be apparent, however, to one skilled in the pertinent art, that the invention may be practiced without these specific details. In other instances, well known features have not been described in detail so as not to obscure the invention. The claims following this description are what define the metes and bounds of the invention.

The present disclosure presents concepts and improvement on the concepts and applications previously disclosed by the same inventor, Alfredo Zolezzi-Garreton, in a United States patent application (U.S. application Ser. No. 13/021,707), which is included in its entirety by reference in the present disclosure. The disclosure describes, among other exemplary applications, an embodiment of the invention where a system implementing the invention is able to disinfect water that contains pathogens. Diverse systems and methods may be designed following the teachings of the invention to carry out many different applications without departing from the spirit and scope of the invention.

### GENERAL CONCEPT

Under usual conditions, the concentration of charge carriers (electrons and ions) in a gas is very low, consequently, a gas is a very good dielectric. In order to acquire any significant electrical conductivity, a gas requires the presence of a high quantity of charge carriers, which can be created through ionization. A gas acquires a steady electric conductivity once there is equilibrium between the origination and disappearance of charges.

The most common method to artificially create plasma is through creating an electric arc between a pair of electrodes under high-voltage. In a gas, the discharge voltage has to reach a given level i.e. breakdown voltage, in order to ionize gas particles. The plasma state may then be maintained through the passage of a sustained electric current though the plasma.

The appearance or threshold of discharges in the gas phase depends considerably on the pressure of the gas. Thus, in the case of a uniform field of breakdown voltage (self-maintained discharge initiation voltage) the threshold is determined by the product of pressure by the distance between the electrodes, according to Paschen's Law. Paschen determined that breakdown voltage is determined by the following equation:

$$V = \frac{a ( pd )}{ln ( pd ) + b}$$

where “V” is the breakdown voltage in Volts, “p” is the pressure in atmospheres, “d” is the gap distance between the electrodes in meters, and “a” and “b” are constants characterizing the particular gas between the electrodes. Thus, in contrast to liquids, which are relatively incompressible, different forms of electric discharge can be implemented in gases by varying the pressure of the gas between the electrodes.

The method, in accordance with the invention, transitions a liquid stream into a gas/liquid biphasic stream, for instance, by subjecting a liquid stream to a pressure drop. The liquid, thus, expands in a sort of vaporization phenomenon obtaining an aerosol i.e. nebulization.

In addition to the latter intrinsic behavior, the nebulization of the liquid may be aided in embodiments of the invention by one or more means for atomizing the liquid. For example, a system implementing the invention may utilize a nozzle, a diaphragm, a hydrodynamic transducer, an acoustic transducer or any other means capable of producing droplets of liquid. Said nebulization facilitates the creation of electric discharges within the fluid. The invention provides control over the ratio of gas-to-liquid, since increasing the latter ratio creates conditions that facilitate electric breakdown, thus promoting the creation of a plasma state.

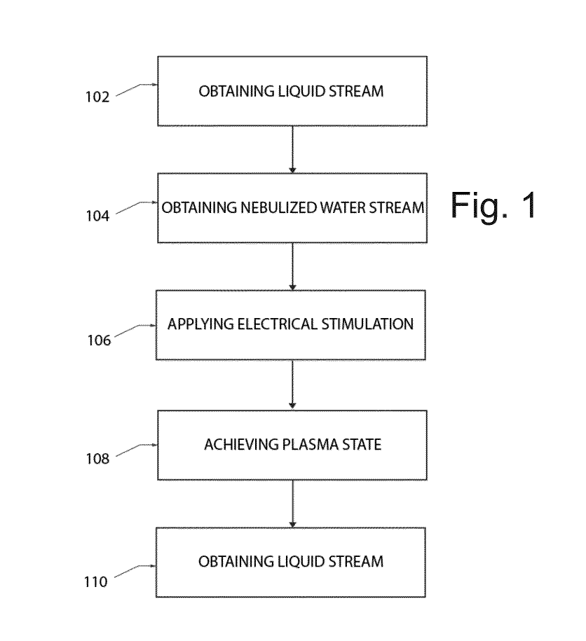

FIG. 1 is a flowchart diagram that represents the overall steps for creating a plasma state in a gas-liquid biphasic medium, in accordance with an embodiment of the invention. At step 102, a system embodying the invention obtains a liquid mixture, feedstock. The feedstock may comprise any number and type of liquids, optionally mixed with one or more diluted, suspended and/or emulsified substances. The composition of the feedstock may be selected by a user for any specific application, using an embodiment of the invention. The feedstock may contain water, electrolytes and any other substance (e.g., oils) that may be targeted for breakdown, synthesis or promotion (e.g., catalysis) in a reaction in accordance with an application of the invention.

At step 104, a system embodying the invention passes the feedstock through a device that transitions the feedstock from a liquid phase into a gas-liquid biphasic state. The latter may be achieved, by passing the liquid through a nozzle, a diaphragm, a hydrodynamic, magnetostrictive or piezoelectric transducer or any other means capable of partially vaporizing a liquid.

For each feedstock, the parameters and means of transitioning the liquid into the gas-liquid biphasic state may be adjusted. For example, the size of the opening of a nozzle, and many other parameters, such as pressure, the hydrodynamic transducer adjustments, or any other parameter, may be adjusted according to the density and/or composition of the feedstock, or any other requirement of a given application for which an embodiment of the invention is used.

In an implementation of the invention, the transition of the feedstock from a liquid to gas-liquid biphasic state may be designed to occur at the passage into a reaction chamber, to which it may be referred as a reactor in this disclosure.

At step 106, the feedstock is passed through a reactor, where the biphasic stream is subjected to a stimulation (e.g. an electric field). At step 108 the stimulation is able to ionize the particles of the gas-liquid biphasic medium, thus, transforming said biphasic medium into plasma. The presence of the plasma particles, the temperature, the pressure, the feedstock composition and other parameters determine the type of the chemical reactions that may take place in the reactor. Plasma state increases the local temperature and pressure and generates other effects such as luminescence, infra-red (IR) radiation and ultraviolet (UV) radiation. Chemical bond breakage and liberation of radicals may follow. For example, if the fluid were water, plasma state would generate ozone and oxygen-hydrogen (OH) radicals, bearing an extremely reactive and oxidative atmosphere, which has important effects on the fluid.

In the reactor, a stable and stationary electric plasma discharge may be realized. These stable characteristics can be measured for each medium, thus making it possible to optimize the burning parameters by collecting data and adjusting the parameters in order to fulfill specific technological tasks, according to the desired application. Given the stability of the burning characteristics, the invention allows one to easily adjust the requirements or power.

Step 108 comprises a plurality of method steps, which may be designed to separate byproducts of the reaction. According to one or more embodiments of the invention, the byproducts comprise one or more gases, liquids and/or solids.

At step 110, the stream of liquid-and-gas feedstock is converted back to a liquid state. However, since other solid and/or gas compounds may result from the reaction that takes place in the reactor, those compounds may be separated thought other means that do not require a conversion to a liquid phase.

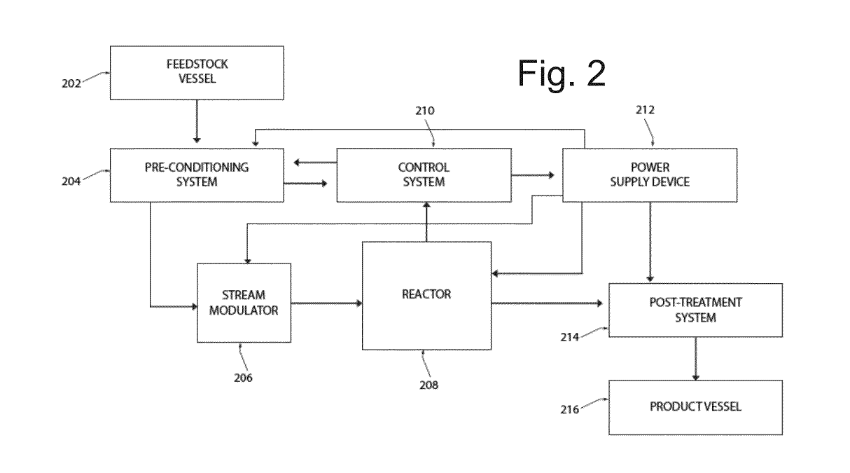

FIG. 2 is a block diagram representing the basic components of a system implementing the invention for applying plasma to a liquid. Block 202 represents one or more feedstock sources (to which it is also referred as feedstock vessel) that may be a tank for storing feedstock and/or preparing a mixture of feedstock. In addition, block 202 may represent a pipeline for continuous feed of feedstock. Feedstock vessel 202 feeds a pre-conditioning system represented by block 204. Feedstock may be transferred from the feedstock source 202 to the pre-conditioning system using pumps, pipelines and any other device required to transport the feedstock.

Block 204 represents one or more pre-conditioning systems that may comprise a heater, a cooling system, a vacuum and/or a compressing device and any other system that may be beneficial for treating the feedstock before treating the liquid with plasma in accordance with embodiments of the invention. An apparatus implementing the invention may easily change the operation parameters such as temperature, pressure, density, concentration and any other parameter, at the pre-conditioning level. The ability of the pre-conditioning system to be configured in many ways enables an embodiment of the invention to provide optimal set of parameters to initiate and maintain a stable plasma discharge for each desired outcome of the application of plasma to the feedstock and/or type of feedstock.

Block 206 represents a stream modulator, where feedstock may be treated with mechanical waves to atomize a liquid and generate a nebulized state. Stream modulator 206 may provide flow control of the feedstock, which may be implemented as a mechanical means for flow control. For example, a stream modulator may be equipped with mechanical (or electromechanical) equipment to pressurize a liquid passing through the stream modulator 206, the liquid may eventually be a mixture that includes one or more gases.

Block 210 represents a data collection, processing and control system. Any of the components of a system embodying the invention may be configured to collect and transmit data to the control system. For example, environment parameters, such as temperature and pressure, may be measured at any stage of operation, and the data collected and processed. Furthermore, the control system may be configured to control any device of the system and use the feedback data to optimize operations. For example, the control system may control the pumps in order to increase or decrease the pressure inside a reactor, in order to optimize the pressure level required by a given chemical reaction, and the flow rate through the reactor, the pre-conditioning system, the post-conditioning system or any other component of a system embodying the invention.

The pressured stream flows through the pre-conditioning system 204 through a high pressure pipeline and stream modulator 206 into a reactor 208, where plasma is produced using one or more means comprising: capacitively, inductively, microwaves or a combination thereof. According one or more embodiments of the invention, a plasma condition is reached in the reactor with a volumetric morphology, which allows for implementing a highly effective plasma treatment system at several scales.

In an embodiment of the invention, the pressure of the medium before the reactor may be, for example, in the range of 1 to 100 bar, whereas the pressure in the reactor may be in the range of 0.1 to 0.01 bar, the pressure after the reactor may be in the range of 0.5 to 4 bar. The measured pressure in a nozzle zone may usually be 0.1 bar and the pressure of a liquid before the reactor may be 100 bar.

The discharge regimes may be adapted to achieve a plurality of results, the following are examples of variants of excitation of the discharge:
The electric discharge may be on a constant voltage from a rectifier through a ballast resistance.
The discharge may be from an energy storage device (e.g. Capacitor) charged to the voltage of breakdown.
The discharge may be generated by an alternating voltage source, pulsed or continuous regime (with a wide frequency range), or direct voltage source in pulsed or continuous mode (e.g., having a frequency of 30-50 kHz). In tested cases, plasma was ignited using pressure (i.e. before the reactor pressure) of 100 bar, and a discharge voltage of 10 KV or above. In stationary regime, the pressure could be lowered to 65 bar or less, and the voltage between the electrodes was between 500 to 4000 V (or above), which depended on the geometry of the chamber. Discharge current was between hundreds of milliamperes (mA) to a few Amperes (A).
The plasma ignition can be achieved using an external coil surrounding the reactor 208 connected to a high frequency alternating power source, to induce electric currents inside the reactor 208.
The plasma ignition can be achieved using an external waveguide and a magnetron surrounding the reactor 208, to induce microwaves inside the reactor 208.

The reactor 208 may comprise a plurality of devices for controlling the environment created inside the reactor. For example, the reactor comprises an emergency dump valve, which may be triggered by a set of security sensors such as manometers, thermometer, vacuum meter or any other sensor. The reactor comprises one or more nozzles for adding reagents inside a reactor.

In order to improve the gas-to-liquid ratio of the nebulized medium, and depending on the application where the disclosed method is utilized, a nozzle, diaphragm, hydrodynamic, magnetostrictive or piezoelectric transducer may be utilized to further enhance the creation of the gas-liquid biphasic mixture. In embodiments of the invention, several different types of plasma can be produced with minimal changes to the reaction chamber. The latter may be achieved, for example, by modifying the operation parameters of the power supply unit (e.g., block 212).

Block 212 represents one or more power supply systems. A power supply 212 may be used to control the electric discharge, it may also be configure to be controlled by the control system 210 in order to adjust the operations parameters for optimal use of a system embodying the invention.

After passing the reactor 208, the stream flows through a narrowing pipeline into one or more post-treatment system represented by block 214. The pressure level after the reactor may be set using the diameter of the narrowing pipeline.

Block 214 represents one or more post-treatment systems comprising one or more means to treat the outlet stream in order to reach any specific operational objective. As described above, the byproducts of the reaction (or reactions) taking place in the reactor may be numerous, and may be characterized by their own state. The post-treatment stage 214 may include any combination of at least one cooling device, at least one compressing device, at least one condensation device and any other device that may be beneficial to any specific implementation of the invention. In the case where the feedstock contains a combination of two or more substances, or since generally the product of the plasma treatment results in a liquid that contains more than one substance, the post-treatment system may comprise several stations. For example, in one embodiment of the invention, post treatment may comprise several post-treatment stations each of which may collect an individual substance from the liquid. The latter may be achieved in the case of substances that possess distinct condensation temperatures by providing multiple condensation stations where each station provides the temperature and/or pressure to allow for the condensation of a target substance or combination thereof.

The products and/or the remaining liquid is/are collected in a product vessel 216. To fully utilize the unused feedstock, tanks 216 and 202 may be connected in a closed loop operation of the system.

The components of a system embodying the invention, as introduced above, may be multiplied and mounted in parallel and/or in a series in order to scale any application to an industrial level. The modularity of the system also allows for one stage to be carried out in one location and liquids and/or gases transported to other locations for use and/or for further treatments.

### Means for Igniting and Maintaining Plasma State in the Biphasic Medium

As described above, the ionization of the biphasic medium is crucial to the initiation and maintenance of the plasma state in implementations of the invention. Accordingly, for each type of feedstock an optimal set of parameters is determined depending on the application. Tests conducted during the reduction to practice of the invention show there is a wide range of parameter combinations that produce a stable plasma discharge inside a reactor. Moreover, for pure liquids it is possible to calculate optimal parameters or estimate them theoretically. The parameters for mixtures may be determined experimentally using an embodiment of the invention.

Embodiments of the invention are equipped to allow for dynamically adjusting treatment parameters in order to achieve a desired effect. Embodiments of the invention may include sensors for determining some characteristics of the treated liquid flow, such as electrical conductivity, temperature, pH, oxidation/reduction potential, or any other parameter that may be measured or assessed, or computed. Embodiments of the invention may utilize the real-time input from the sensors to adjust the parameters in order to keep the apparatus operating at a preset regime of operation. For example, in order to maintain a preset burning temperature, the temperature measured within the reactor (or within any portion of the apparatus as the case may be), the power source may be regulated, for example, to modify the level of stimulation. In other instances, a magnetic or electromagnetic field may be applied to the reactor for magnetic confinement of the plasma discharge. Yet, in other instances the position of the electrodes may be mechanically and dynamically changed a means to control the ignition and maintenance of plasma.

The ionization of the biphasic medium depends on the size of the droplets of liquid suspended in the gas phase. Sonic or ultrasonic electromechanical equipment may be used in the stream modulator 206 in one or more embodiments of the invention. This electromechanical equipment can work under cavitation regime or under suppressed cavitation regime. Provided that the feedstock may contain, water one or more oils, solid particles such as fibers, organic and/or mineral matter, volatile compounds, the application of sonic and/or ultrasonic may result in promoting one or more physical and/or chemical reactions including nebulizing, intensive mixing, oxidation and any other inherent reaction caused by the application of sonic waves.

Due the generation of shear forces caused by ultrasonic fields, it may be possible to produce oxidizing agents that may be able to eliminate microorganisms, from precursors comprised in the treated liquid or added to it, in gaseous or liquid phase, in the reaction chamber or at any previous stage. Furthermore, ultrasonic waves are able to affect said microorganisms by itself.

Moreover, the reactor 208 incorporates one or more nozzle, hydrodynamic, piezoelectric or magnetostrictive transducer at the inlet, for controlling the income of the feedstock into the reactor, with the aim of optimizing the volumetric plasma generation, for instance, by creating a turbulent biphasic flow which occupies the entire internal volume of the reactor, also having intensive mixing.

In an embodiment of the invention, a liquid stream is forced through a nozzle, from a high-pressure zone to one where the pressure is lower than the vapor pressure of said liquid at the local temperature. The liquid stream is accelerated, then, at the expansion zone of the nozzle the liquid is partially vaporized, as the pressure is lower than the vapor pressure. The flashing phenomenon is an abrupt adiabatic phase change, so it can be seen as a discontinuity in the field, and occurs on the surface of a liquid core that rises from the nozzle through an evaporation wave process.

Plasma state is generated in the reactor chamber, according to the invention, by applying an electric field in one or more of a plurality of manners disclosed in the following.

Plasma state is generated in the reactor chamber by applying an electrical field in the conditioned nebulized biphasic medium capacitively coupled plasma, using at least two electrodes, positioned in the interior and/or exterior of the reaction chamber. A dielectric barrier for creating a dielectric barrier discharge may be utilized.

Plasma state is generated in the reactor chamber by applying an electrical field in the conditioned nebulized biphasic medium inductively, using external coils to create electromagnetic fields the magnitude and direction of which may be dynamically controlled.

Plasma state is generated in the reactor chamber by applying an electrical field in the conditioned nebulized biphasic medium using microwaves. The latter is achieved using a magnetron and an external wave guide.

Plasma state is generated in the reactor chamber by applying Laser or Maser radiation to the conditioned nebulized biphasic medium.

In the implementation of this invention, in particular for high throughput and high residence time in the reactor 208, one or more embodiments include the use of at least two internal electrodes, one of which at the inlet and another of which at the outlet of the reactor, and as the case maybe one or more external electrodes. One or more external electrodes may be used for producing a dielectric barrier type electric discharge in the biphasic medium, where the reactor walls are used to act as a dielectric barrier. In some applications the external electrode(s) may be fabricated using a metallic sheet and/or alternatively using a conductive paint that is applied to the exterior and/or the interior surface of the reactor. The latter configuration of electrodes allows for generating a highly volumetric plasma discharge inside the reactor.

Moreover, using external electrodes in addition to internal electrodes allows for establishing a plasma channel in reactors of extended length without requiring a proportional increase of voltage, as opposed to using only internal electrodes. In an apparatus that embodies the latter configuration, igniting plasma occurs as a progressive process, where plasma is ignited at the inlet side of the reactor, then under the influence of the dielectric discharge, plasma is progressively ignited toward the outlet until it reaches the electrode at the outlet side of the reactor. Once plasma has been ignited and there is sufficient ionized gas to support the current passage between the inlet electrode and the outlet electrode, the dielectric effect may be sustained or may be stopped to reduce the energy consumption.

A few examples of implementation of the teachings of the invention are described below in detail to illustrate how some of the configurations described above may be implemented in a plurality of applications. The examples show the breadth of the scope of the teachings of the invention, and may not be construed as the limiting the implementations of the teachings of the invention.

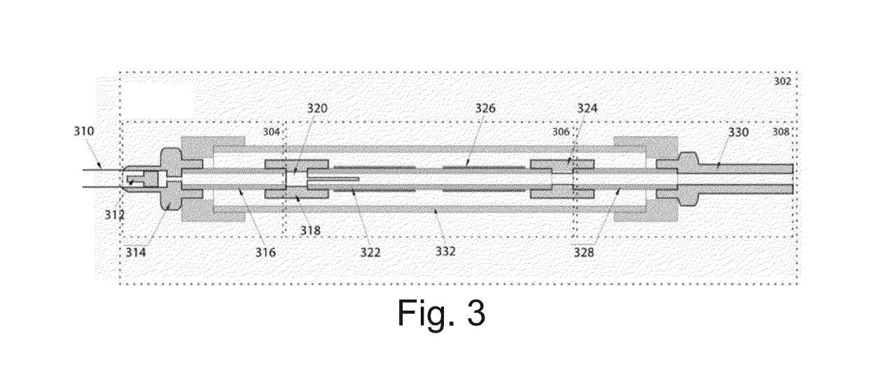

FIG. 3 represents a preferred embodiment of the invention intended for the treatment of water, at a rate of 4-7 liters per minute, in which the reaction chamber 302 is a tubular vessel having a length in the range of 20 cm to 60 cm, 2-5 cm of external diameter and 0.5-2 cm of internal diameter. The vessel can be made of any suitable dielectric material, preferably borosilicate glass, other components can be made of any conducting material.

The water to be treated is pressurized inside a pipeline 310 up to 60-70 bar and fed into the reaction chamber. The pipeline is connected to the reaction chamber 302 through a nozzle 314. The water stream abruptly accelerates reducing the pressure inside the reaction chamber to approximately 0.08 bar. Inside the nozzle 314 there is a flow deflector 312, used to avoid the formation of a laminar flow of the liquid before passing through the nozzle. The preferred material for both the nozzle and the flow deflector is stainless steel, however any suitable material may be utilized.

The reaction chamber 302 of this embodiment of the invention is a continuous tubular vessel comprising three consecutive sections each of which serves a specific function i.e., evaporation section 304, plasma section 306, and condensation section 308.

The evaporation section comprises a nozzle 314 and an evaporation chamber 316. Its function is to accelerate the water stream entering the reaction chamber, which creates a vacuum condition inside the reaction chamber. Due to the pressure drop caused by the acceleration of the liquid stream, the water passing through the nozzle 314 partially evaporates, creating a continuous gas-liquid biphasic stream that occupies the whole internal volume of the reaction chamber, and passing at high velocity. According to the invention, the gas-liquid biphasic stream is a suitable medium for generating plasma.

The evaporation section 304 is connected to the plasma section 306 by a conductive joint 318, which has a tubular shape with the same internal diameter than that of the evaporation section, and can be made of any conducting material, preferably stainless steel.

The plasma section 306 comprises an electric discharge chamber 322, a first internal electrode 320, a second internal electrode 324 and at least two external electrodes 326. In this section, an alternating electrical field is applied by means of the internal and external electrodes, for ionizing the gas-liquid biphasic medium, thus, generating plasma.

The first internal electrode 320 is located inside the conductive joint 318 between the evaporation section and plasma section, in direct contact with biphasic stream. The latter electrode has a preferred length of 5 cm and a thickness of about 0.1 cm for avoiding drag effect on the high velocity biphasic flow. It may be made of any conducting material, preferably tungsten.

The second internal electrode 324 is located at the exit of the plasma chamber. It has a tubular shape the inner diameter of which is the same as that of the electric discharge chamber 306, therefore, it does not obstruct the passage of the ionized particles, which avoids and/or minimizes drag effect. The specific shape of the electrode further allows the formation of plasma in the entire internal volume of the reaction chamber adjacent to second electrode, ensuring that all particles are subjected to the ionization process. The second electrode has a preferred length of 1-3 cm and can be made of any conducting material, preferably stainless steel with a tungsten coating. The second external electrode 324 also connects the plasma section 306 and the condensation section 308.

The external electrodes 326 are located in direct contact with the external surface of the electric discharge chamber 322, which acts as a dielectric barrier between internal and external electrodes. A preferred embodiment of the invention utilizes two (2) external electrodes. The latter electrodes may be made of any conducting material. In one embodiment of the invention, the external electrodes are fabricated by applying a silver plating over the electric discharge chamber, which silver layer is then covered by a layer of copper. Metal application may be carried using any suitable process including electrolytic copper, spraying or any other means for applying a layer of metal.

The application of a conductive coating as external electrodes allows generating a homogeneous dielectric barrier effect, with no additional components and avoiding a gap between the electrodes and the dielectric barrier. The implementation of two non-continuous external electrodes avoids the formation of a continuous current, thus, improving the efficiency of the power supply.

The external electrodes 326 create a dielectric barrier discharge effect, which allows increasing the length of the chamber 302 without the need of proportionally increasing the operation voltage. As a result, the time of residence of the treated water can be efficiently increased, enhancing the effects of the treatment.

The condensation section 308 comprises a condensation chamber 328 and an output connector 330. The condensation chamber separates the condensation process from the plasma zone, thus ensuring that all particles are ionized inside the plasma zone. The ionized particles stream is decelerated and condensed at the end of the condensation chamber 328 and inside the output connector 330. The condensation section allows maintaining a vacuum condition in a volume larger than that required for ensuring that the plasma is formed stably and homogeneously in the plasma section.

The evaporation and condensation chambers are also security sections where there is only gas-liquid biphasic medium with no generation of plasma. This allows said chambers to act as non-conductive isolation barriers. This condition improves the electrical insulation between the electrodes and the rest of the system, creating a floating voltage system. The latter configuration increases the electrical safety of the system, and eliminates losses of currents toward the ground.

The three sections of the reaction chamber are placed inside an external chamber 332 filled with an insulation liquid (e.g. transformer oil), for ensuring the insulation of the equipment. The external chamber 332 also avoids the formation of corona discharge and surface plasma outside the chamber, and enhances structural properties of the reaction chamber.

For generating the electric discharge, the preferred embodiment of the invention uses a high voltage, high frequency power supply. The power supply must be able to produce a high voltage output in open circuit condition, so as to be able to generate enough electrical field intensity to ionize the gas-liquid biphasic stream, in the order of 15 KV amplitude at 50 KHz. When the plasma is established the conductivity of the medium inside the reaction chamber increases, then, the generated plasma draws more power, increasing the current. In this stage, the power source must have a current source behavior, with a stable output voltage in the 5 KV range.

To achieve said eclectic discharge, this embodiment of the invention uses a 50 KHz inverter configuration with a resonant stage connected to an elevator transformer.

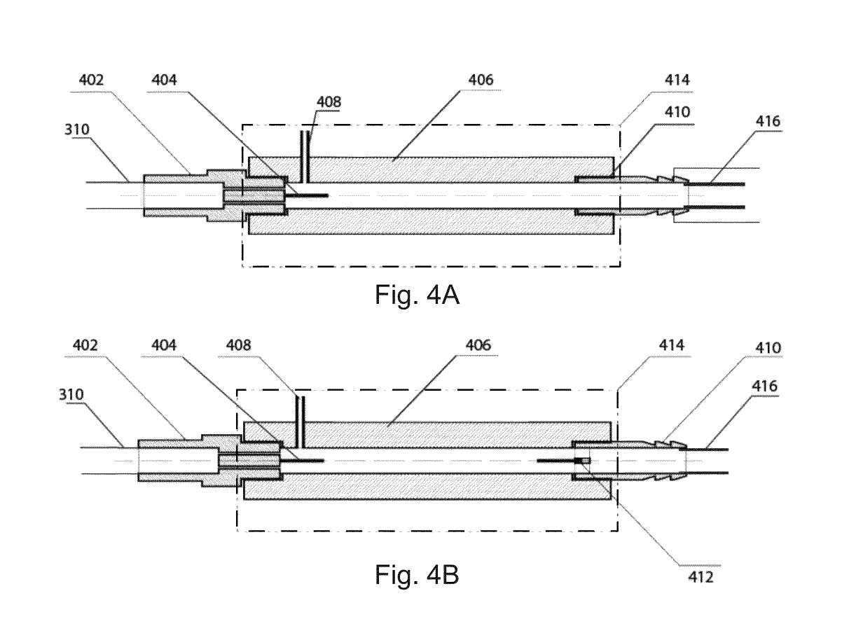

FIG. 4A shows a cut-through representation a portion of a system for transforming a liquid stream into plasma, in accordance with an embodiment of the invention. The liquid stream in the pipeline 310 flows from a zone of high pressure into a low-pressure zone inside a reaction chamber, to which it is referred as plasma chamber 406. The transition from high to low pressure transforms the liquid stream in the pipeline 310 into a nebulized stream inside the plasma chamber 406. Electrodes 404 and 410 are located inside the plasma chamber 406 according to the needs of the intended application. Inlet electrode 404 includes a tungsten insert. The inlet electrode is shaped to maximize the electric discharge while facilitating fluid dynamics. In this embodiment, outlet electrode 410 is also connected with the non-conductive outlet pipeline. The inlet and outlet electrodes are connected to a source of voltage that provides ignition and maintenance of the stationary plasma discharge.

Valve 408 allows for adding precursors (e.g., solid, liquid and/or gaseous compounds), directly into the plasma chamber during operation. Valve 408 may also be used in an apparatus embodying the invention to control the pressure inside the chamber.

After passing the discharge zone 414, the gas-liquid biphasic stream flows into a narrowing zone of pipeline 416 where it condenses back to liquid state.

FIG. 4B shows a cut-through representation of an embodiment of the invention. The liquid in the pipeline 310 flows from a zone of high pressure into a low-pressure zone in plasma chamber 406. The transition from high to low pressure transforms the single-phase stream in the pipeline 310 into a gas-liquid biphasic stream inside the plasma chamber 406. Electrodes 404 and 412 are located inside the plasma chamber 406 according to the needs of the intended application. Inlet electrode 404 includes a tungsten insert with a geometry designed specifically to improve electric discharge and flow. Outlet electrode 412 includes a similar tungsten insert with a specific geometry to improve electric discharge. The electrodes are connected to a source of voltage that provides ignition and maintenance of the stationary plasma discharge. After passing the discharge zone 414, the nebulized stream flows into a narrowing zone of pipeline 416 where it condenses back to a liquid stream. Valve 408 enables a system for adding precursors, in solid, liquid or gaseous phase, into the plasma chamber 406. Valve 408 allows for adding precursors (e.g., solid, liquid and/or gaseous compounds), directly into the plasma chamber during operation.

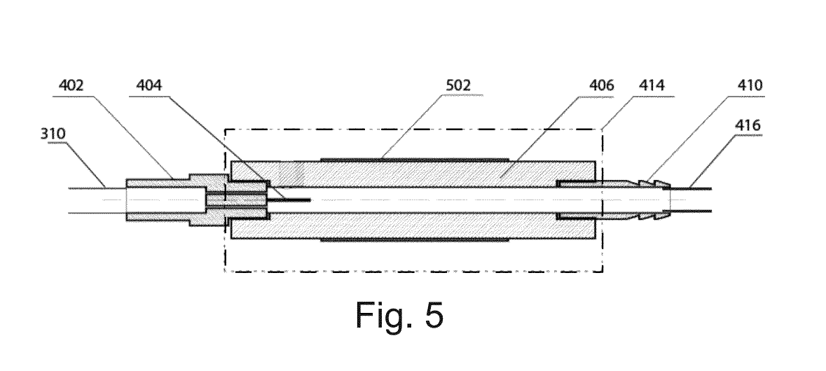

FIG. 5 shows a cut-through representation of a portion of another embodiment of the invention. The liquid in the pipeline 310 flows from a zone of high pressure into a low-pressure zone in plasma chamber 406. The transition from high to low pressure transforms the single-phase stream in the pipeline 310 into a gas-liquid biphasic stream inside the plasma chamber 406. Electrodes 404 and 410 are positioned inside plasma chamber 406 according to the needs of the intended application. Inlet electrode 404 includes a tungsten insert with a specific geometry to improve electric discharge and flow. In this embodiment outlet electrode 410 is connected to the non-conductive outlet pipeline 416. This embodiment includes an external electrode 502 to work with the dielectric reactor wall in a dielectric barrier discharge regime. The electrodes are connected to a source of voltage that provides ignition and maintenance of the stationary plasma discharge. During operation, electrical discharge occurs between internal electrodes and the external electrode that promote charges accumulation in the interior wall of the reactor and its discharge over the saturation limit in a continuous way. After passing the discharge zone 414, the plasma channel flows into a narrowing zone of pipeline 416 where it is converted into a liquid stream.

The invention provides the basic methods and apparatus to carry out a plurality of applications, each of which may be designed to reach a specific goal. The goals of transforming a liquid stream into plasma are numerous, and each specific application may be designed to destroy microorganisms in the treated liquid, or to produce a chemical reaction leading to breakdown of one or more substances. In other embodiments the goal may be the synthesis of new products starting from initial products present in the feedstock. Yet, in other embodiments the goal may be a combination of both breakdown of one set of compounds while synthesizing other products. One with ordinary skills in one or more areas of expertise such as plasma physics, engineering, chemistry and biochemistry would recognize that by providing the means to generate plasma in a highly controllable environment, the invention opens the way to numerous applications whose goal may be to breakdown some substances, for example, in order to remove toxins from waste water, the synthesis of molecules such as the formation of molecular hydrogen or a combination of both.

### Method and Apparatus for Sanitizing Water

The invention provides a method and system for disinfecting water. The conditions created inside the reactor in the presence of plasma (see above description) in combination with the transformation of a liquid into plasma, following the invention's teachings, provide an effective method for destroying biological agents in water that may pose a danger to a consumer.

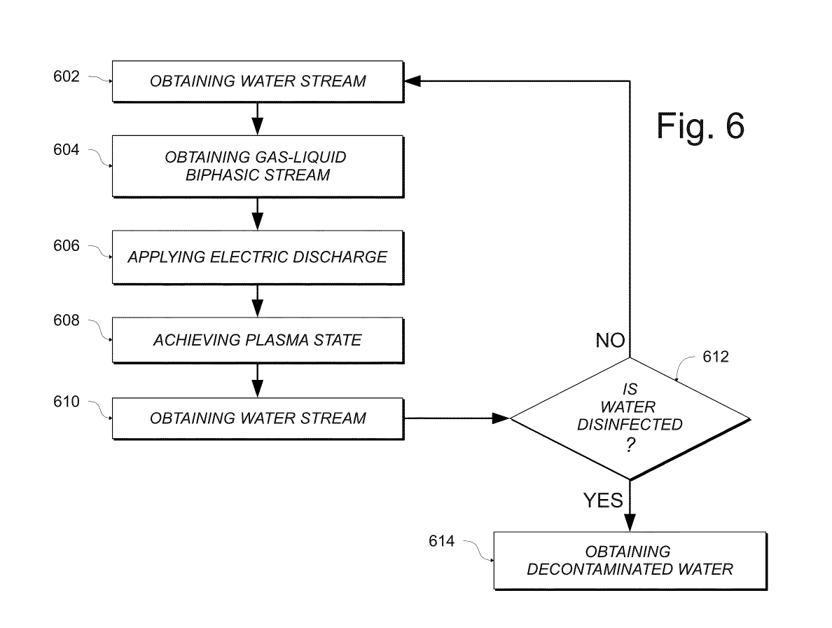

FIG. 6 is a flowchart diagram representing steps of disinfecting water in accordance with an embodiment of the invention. At step 602, a water supply potentially containing harmful biological agents is brought to a disinfection system embodying the invention. Step 602 may involve other steps of pretreatment comprising filtering, decanting, ionically separating one or more compounds, mixing with chemicals, separating one or more compounds using flocculation and/or any other step of pretreatment.

At step 604, the liquid stream is converted into a gas-liquid biphasic stream by a stream modulator that may include piezoelectric or magnetostrictive electroacoustic transducers fed by an electroacoustic generator, and the different means, nozzle, hydrodynamic, piezoelectric or magnetostrictive transducer that incorporates the inlet of the reactor for conditioning the fluid entering the electrical discharge zone.

At step 606, an electric discharge is applied to the gas-liquid biphasic stream inside the reaction chamber, for example, by passing a high voltage electric current through the electrodes. At step 608, the particles of the biphasic stream are ionized by the electric discharge.

Multiple water disinfecting effects are generated by the creation of plasma. Among the disinfecting factors are: ultraviolet radiation (UV), infrared radiation (IR), ozone and the shock of ultrasonic vibrations. For instance, using the parameters specified above, UV with wavelength about 320 nm and IR with wavelength 840 nm are generated in the plasma chamber.

Table 1, below, lists a summary of disinfectants produced in the presence of plasma, and the expected effects of the application of disinfectants on biological agents in the water.

TABLE 1

| Agent | Effect | Disinfection Result |
|---|---|---|
| UV | Disrupting DNA | disrupting microorganism reproduction |
| Radi- | and Blocking | killing microorganisms through blocking |
| ation | protein synthesis | expression of proteins |
| IR | Raising | killing microorganisms through coagulation |
| Radi- | temperature | of proteins (e.g., enzymes) |
| ation | | enhancing the efficiency of other |
| | | disinfectants |
| Ultra- | Mechanical | Mechanical destruction of microorganisms |
| sound | shearing | |
| Ozone | Oxidation | Breaking cell wall of microorganisms |
| | | affecting nucleic acids of microorganisms |

Electrical discharges may create several oxidizing agents that are known to have disinfecting effects, directly in the treated medium, from precursors present in the treated liquid and/or injected in liquid or gaseous phase, before exiting the plasma zone.

Due the hydrodynamic effects caused by the means of generating the biphasic medium and conditions arising during plasma state, the oxidizing agents come extremely closely to the target biological contaminants.

Rigid UV light (with short wavelength) is most effective for destruction of biological agents. As the pressure of the electric field increases, the wavelength of UV of 200 nanometers and lower tends to steadily decrease. Also, in the latter case, a high concentration of ozone is generated in the plasma chamber.

Ultrasound (US) with frequency 15 to 40 kHz is able to deactivate biological agents. In this case, incoming water moves through a hydrodynamic transducer into the plasma chamber. The hydrodynamic transducer may be preliminary adjusted to the above range of frequencies and may also play a function of an entrance nozzle to the plasma chamber.

Embodiments of the invention may use electrodes made from such metals as silver or titanium, which may increase the antibacterial properties of the treatment. The introduction of rod-like electrodes in a discharge zone results in a saturation of water by ozone. Due to its highly oxidizing properties and effect on the biochemistry of biological agent, ozone is extremely effective for the inactivation of bacteria and many kinds of microbes.

At large amounts of electric current of the discharge, intense radiation in a wide range of wavelengths from ultraviolet to infrared is observed. The latter promotes the formation of chemically active particles in plasma and in a liquid. By varying electric parameters it is possible to control the wavelength of the emitted radiation, thus generating a wide spectrum of ultraviolet radiation in the range of 300 to 600 nanometers. The latter also favors water sanitization, since ultraviolet penetrates an organism cell wall disrupting its genetic material.

The hydrodynamic transducer generates an ultrasonic field in the medium, which provides an accelerated mass transfer of the plasma discharge products (ozone, atomic oxygen, oxygen ions and other oxidizers) to the microorganisms and pollutants. This way, the plasma discharge products affect the microorganisms and pollutants in a short amount of time and the sanitization is efficient.

A reactor in accordance with the invention may, in addition to producing each of the disinfecting agents (e.g., ozone, ultraviolet and ultrasonic waves etc.) alone, also implement two or more of the latter mechanisms simultaneously. A combination of two or more of these agents is even more effective at sanitizing water, since the effects are cumulative.

Ultraviolet (UV) light is the spectrum of electromagnetic radiation within the scope of 10 nm to 400 nm. The possibilities of using UV light for water disinfection have been known for several decades. UV light penetrates the cell body, disrupts Deoxyribonucleic Acid (DNA) and Ribonucleic Acid (RNA), which support the storage and expression of all genetic information in an organism, thus preventing reproduction or killing the cells. UV treatment does not alter water chemically. Nothing is being added except energy.

Ozone is produced when oxygen (O₂) molecules are dissociated by an energy source into oxygen atoms and subsequently collide with an oxygen molecule to form an unstable gas, ozone (O₃), which is used to disinfect water. Ozone is generated onsite because it is unstable and decomposes to elemental oxygen in a short amount of time after generation; it is very strong oxidant and bactericide. The mechanisms of disinfection using ozone include:
Direct oxidation/destruction of the cell wall with leakage of cellular constituents outside of the cell.
Reactions with radical by-products of ozone decomposition.
Damage to the constituents of the nucleic acids (purines and pyrimidines).

When ozone decomposes in water, free radicals, such as hydroperoxyl (HO₂) and hydroxyl (OH⁻) are formed and have great oxidizing capacity, which play an active role in the disinfection process. It is generally believed that the bacteria are destroyed because of protoplasmic oxidation resulting in cell wall disintegration.

Main advantages of ozone disinfection are that Ozone is more effective than chlorine in destroying viruses and bacteria, and there are no harmful residuals that need to be removed after ozonation because ozone decomposes rapidly. After ozonation, there is no regrowth of microorganisms, except for those protected by the particulates in the waste water stream.

At step 610 the stream subjected to plasma condition is brought back to a water solution, as described above. A test for the effectiveness of the treatment may be conducted at step 610. If the water is found to have been disinfected to a satisfactory level 612, the water is then piped out of the system (e.g., disinfection station) at step 614, otherwise the water may optionally be pumped back into the reactor for further treatment.

Tests performed by National Sanitation Foundation on a system embodying the invention, following testing protocols P231 and P415, have demonstrated the high effectiveness of this method for eliminating microbial agents in water, achieving total elimination of microorganisms in the treated water samples.

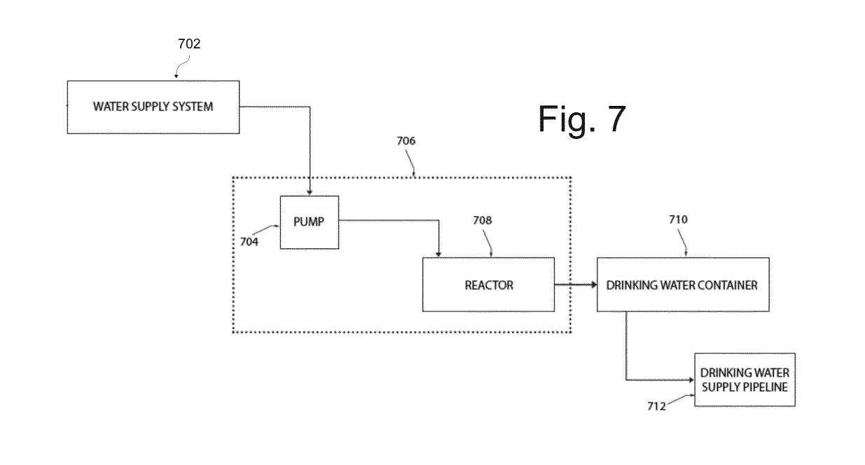

FIG. 7 is a block diagram representing components of a system embodying the invention for providing water sanitization. Block 702 represents a source of fresh water that is potentially (or suspected of being or known to be) contaminated with biological agents. Such a source may be part of a water network, the purity of which may have been compromised purposefully (e.g., as a result of a terrorist attack), accidentally (e.g., breakage in the sewage system that spills over to the fresh water supply), or naturally such as a well, lake or river the water of which may not meet the consumption standards.

The system may utilize a plurality of apparatuses embodying the invention to increase capacity of water treatment. Block 706 represents a system for dividing the flux of water from one or more sources of water to supply a plurality of apparatuses embodying the invention. For example, water may be transported over long distances and combined with a network of water distribution (e.g., canals, hoses, tubes etc.) to carry the water to one or more treatment stations. Block 706 represents a set of components of the system embodying the invention that carries out the method steps described in FIG. 6. The apparatus may include one or more pumps (e.g., block 704).

Block 708 represents a reactor where plasma is created, thus producing one or more disinfecting agents that affect living organisms. One or more reactors may be mounted in parallel and/or series in order to reach a target treatment capacity and/or disinfection level.

An apparatus embodying the invention may include one or more heat exchangers to bring the temperature of the water to a desired or required output temperature. The output temperature may necessary for delivery to later stage of the water supply system.

Block 710 represents water storage (e.g., water container, open space water storage or any other means for storing water before consumption). Treated water may be checked for disinfection efficiency. Water (or a portion thereof) that has been submitted to a plasma treatment may be returned in a closed loop to the reactor 708 in order to further sanitize it. For example, a closed loop circuit may be designed between any of the system's components downstream from the reactor with any of the components upstream of the reactor.

Potable water may be distributed to consumers by a drinking water supply pipeline 712. The treated water may be distributed by a grid and/or in a standalone manner. For example, an apparatus embodying the invention may be portable and self-reliant for energy and is capable of working in a remote location to provide potable water.

Several embodiments of the invention may be implemented for humanitarian purposes. For instance, in locations where water sources have high content of pathogens, a water treatment system comprising the present method of disinfecting water would be able to provide safe drinking water to a community, at a high efficient rate in terms of the resources needed for its operation. Said system would have additional features such as heavy-duty operation regime, high autonomy and reliability.

Thus a method, apparatus and system of applying plasma particles to a liquid by transforming a liquid stream into a gas-liquid phase medium and igniting plasma in the medium, which produces numerous effects on the treated liquid. The invention may be implemented in several applications. A preferred example is a versatile water sanitization system, which can be implemented from small units for disinfecting water at a household scale, up to large industrial application scale.

What is claimed is:

1. A method of treating a liquid with plasma particles comprising:
obtaining a first continuous stream of liquid;
vaporizing at least a portion of said liquid to produce a biphasic stream within a reaction chamber;
igniting a plasma state to produce plasma particles within said biphasic stream by igniting capacitively coupled plasma using the application of an electric field that exceeds the breakdown voltage threshold of said biphasic stream, and maintaining said plasma state between at least two electrodes inside said reaction chamber; and
condensing said biphasic stream into a second continuous liquid stream.

2. The method of claim 1, wherein said step of igniting said capacitively coupled plasma further comprising using at least two internal electrodes, and at least one external electrode, having a dielectric barrier between said at least two internal electrodes and said at least one external electrode.

3. The method of claim 1, wherein said step of vaporizing further comprises transitioning said liquid from a high-pressure zone into a lower pressure zone using a nozzle.

4. The method of claim 1, wherein said step of vaporizing further comprises transitioning said liquid through a diaphragm.

5. The method of claim 1, wherein said step of vaporizing further comprises submitting said liquid to an acoustic stimulation in a stream modulator.

6. The method of claim 5, wherein said step of vaporizing further comprises utilizing a magnetostrictive transducer.

7. The method of claim 5, wherein said step of vaporizing further comprises utilizing a piezoelectric transducer.

8. An apparatus for treating a liquid with plasma particles comprising:
means for obtaining a first continuous stream of liquid;
means for vaporizing at least a portion of said liquid to produce a biphasic stream within a reaction chamber;
means for igniting a plasma state to produce plasma particles within said biphasic stream, comprising at least two internal electrodes inside said reaction chamber for igniting capacitively coupled plasma by applying an electric field that exceeds the breakdown voltage threshold of said biphasic stream and for maintaining said plasma state; and
means for condensing said biphasic stream into a second continuous liquid stream.

9. The apparatus of claim 8 further comprises at least one external electrode, having a dielectric barrier between said at least two internal electrodes and said at least one external electrode.

10. The apparatus of claim 8, wherein means for vaporizing further comprises a nozzle for transitioning said liquid from a high-pressure zone into a lower pressure zone.

11. The apparatus of claim 8, wherein said means for vaporizing further comprises a diaphragm.

12. The apparatus of claim 8, wherein said means for vaporizing further comprises an acoustic waves generator for stimulating said liquid in a stream modulator.

13. The apparatus of claim 12, wherein said mean for vaporizing further comprises a magnetostrictive transducer.

14. The apparatus of claim 12, wherein said means for vaporizing further comprises a piezoelectric transducer.

15. A method of treating a liquid with plasma particles comprising:
obtaining a first continuous stream of liquid;
vaporizing at least a portion of said liquid to produce a biphasic stream within a reaction chamber;
igniting a plasma state to produce plasma particles within said biphasic stream by igniting inductively coupled plasma using an alternating magnetic field within said biphasic stream; and
condensing said biphasic stream into a second continuous liquid stream.

16. An apparatus for treating a liquid with plasma particles comprising:
means for obtaining a first continuous stream of liquid;
means for vaporizing at least a portion of said liquid to produce a biphasic stream within a reaction chamber;
means for igniting a plasma state to produce plasma particles within said biphasic stream, comprising means for generating an alternating magnetic field within said biphasic stream to ignite inductively coupled plasma; and
means for condensing said biphasic stream into a second continuous liquid stream.

17. A method of treating a liquid with plasma particles comprising:
obtaining a first continuous stream of liquid;
vaporizing at least a portion of said liquid to produce a biphasic stream within a reaction chamber;
igniting a plasma state to produce plasma particles within said biphasic stream by igniting microwave excited plasma by applying microwaves to said biphasic stream; and
condensing said biphasic stream into a second continuous liquid stream.

18. An apparatus for treating a liquid with plasma particles comprising:
means for obtaining a first continuous stream of liquid;
means for vaporizing at least a portion of said liquid to produce a biphasic stream within a reaction chamber;
means for igniting a plasma state to produce plasma particles within said biphasic stream comprising means for generating microwaves to apply microwaves excited plasma to said biphasic stream; and
means for condensing said biphasic stream into a second continuous liquid stream.

19. A method for sanitizing a water source contaminated with biological agents, comprising:
obtaining a water from a water source;
vaporizing at least a portion of said water to obtain a vapor and liquid stream;
igniting plasma state to produce plasma particles in said vapor and liquid stream; and
condensing said vapor and liquid stream into an output water stream.

20. An apparatus for sanitizing a water source contaminated with biological agents, comprising:
means for obtaining a water from a water source;
means for vaporizing at least a portion of said water to obtain a vapor and liquid stream;
means for igniting plasma state to produce plasma particles in said vapor and liquid stream by igniting capacitively coupled plasma using an electric field that exceeds the breakdown voltage threshold of said vapor and liquid stream; and
means for condensing said vapor and liquid stream into an output water stream.
